(19) United States
(12) Patent Application Publication
(10) Pub. No.: US 2012/0232162 A1
(43) Pub. Date: Sep. 13, 2012

(54) USE OF LUTEIN CONTAINING COMPOSITIONS TO IMPROVE CERTAIN ASPECTS OF MEMORY

(76) Inventors: Regina Goralczyk, Grenzach-Wyhlen (DE); Hasan Mohajeri, Egg b. Zurich (CH); Annis O. Mayne-Mechan, Mohlin (CH)

(21) Appl. No.: 13/505,854

(22) PCT Filed: Nov. 5, 2010
(86) PCT No.: PCT/EP10/66949

§ 371 Date: May 3, 2012

Related U.S. Application Data
(60) Provisional application No. 61/272,823, filed on Nov. 9, 2009.

(51) Int. Cl. A61K 31/047; A61P 25/28; C07C 35/21

(52) U.S. Cl. 514/729; 568/816

(57) ABSTRACT

This invention relates to a method of enhancing an aspect of memory in a healthy individual, wherein the aspect of memory is selected from the group consisting of: associative memory, spatial memory and memory under stress comprising: administering a composition consisting of: a) an effective amount of either lutein or the combination of lutein and zeaxanthin; and b) an appropriate carrier; and observing the enhanced associative memory, spatial memory or memory under stress.

### FIELD OF THE INVENTION

The present invention relates to a novel nutraceutical composition or food compositions comprising lutein and/or zeaxanthin to improve certain selective memory functions, such as associative learning, associative memory, learning and memory under stress, and spatial (place) learning.

### BACKGROUND OF THE INVENTION

There is an increasing interest in the development of nutraceutical compositions that may be used to improve learning, memory and alertness, in both elderly and young people.

Lutein has been described as having the ability to ameliorate cognitive decline. See, for example US 2006/0205826 (Romero et al), where it is suggested that lutein could protect the hippocampus in mice with diabetes as it acts as an antioxidant.

WO 2006/116755 (Trustees of Tufts College) reports a synergistic effect of docosahexaenoic acid (DHA) on lutein absorption and transport to the brain after chronic treatment (4 months). This combination can be used to prevent or treat cognitive impairment or dementia, and to prevent memory loss as measured by verbal fluency, word lists, and the MIR (Memory in Reality) Apartment test. Administration of lutein alone only resulted in a significant improvement in the Verbal Fluency test; it did not result in improvements in any of the other cognitive- or mood assessment-tests administered.

Most studies of carotenoids or other natural products which are potential cognitive enhancers involve prevention of cognitive decline in people who already have at least the first stage of dementia (such as short-term memory impairment) or later stages (such as Alzheimer's Disease). There is not much known about lutein or the combination of lutein's and zeaxanthin's ability to enhance selective aspects of memory in healthy populations.

### DETAILED DESCRIPTION OF THE INVENTION

It has been found, in accordance with this invention that lutein, either administered as a sole active ingredient or in combination with zeaxanthin, can boost selected aspects of memory, such as associative learning and memory, learning and memory under stress and spatial learning and memory.

Thus, one aspect of this invention is a method of enhancing an aspect of memory in a healthy individual, wherein the aspect of memory is selected from the group consisting of: associative memory, spatial memory and memory under stress comprising:
administering a composition consisting of: a) an effective amount of either lutein or the combination of lutein and zeaxanthin; and b) an appropriate carrier;
and observing the enhanced associative memory, spatial memory or memory under stress.

Another aspect of this invention is a method of enhancing learning and memory under stress comprising:
administering a composition consisting of: a) a stress-related learning- and memory-enhancing effective amount of either lutein or the combination of lutein and zeaxanthin; and b) an appropriate carrier;
and observing the enhanced learning and memory under stress.

Another aspect of this invention is a method of enhancing spatial learning and memory comprising:
administering a composition consisting of: a) a spatial-related learning- and memory-enhancing effective amount of either lutein or the combination of lutein and zeaxanthin; and b) an appropriate carrier;
and observing the enhanced spatial learning and memory.

Another aspect of this invention is the use of lutein or the combination of lutein and zeaxanthin as the sole active compounds to enhance a) associative learning and memory, b) learning and memory under stress or c) spatial learning and memory. Yet another aspect of this invention is the use of lutein or the combination of lutein and zeaxanthin as the sole active compound(s) which are active in respect to enhancing memory, to make a nutraceutical or functional food which enhances a) associative learning and memory, b) learning and memory under stress or and/or c) spatial learning and memory.

Another aspect of this invention is a kit for use by a person wishing to enhance their associative learning or memory, their learning or memory under stress, or their spatial learning or memory. The kit comprises a dosage form of either lutein or the combination of lutein and zeaxanthin, and optionally instructions for use. Additionally and optionally, there may be separate dosage forms of other active ingredients.

### BRIEF DESCRIPTION OF THE FIGURES

FIG. 1 is a graph illustrating the neuroprotective effect of lutein in a glutamate neurotoxicity paradigm.

FIG. 2 is a schematic presentation of the IntelliCage and its functional features.

FIG. 3 shows the place error rate (percentage of visits to incorrect corners). This effect was observed from the start of the module and was maintained throughout the first 12 h (active phase) and demonstrated that treatment with lutein resulted in a higher level of attention and an improved memory performance.

FIG. 4 shows the side error rate (percentage of nosepokes at the incorrect side of the correct corner). Lutein induced a significantly lower percent error rate compared with vehicle-treated controls.

FIG. 5 shows that lutein-treated mice solved the task to find the correct place to access water under stressful conditions significantly better than control mice.

FIG. 6 shows that supplementation of lutein to animal feed resulted in a significant improvement of mice using spatial cues to learn to find and memorize the location of water.

### DEFINITIONS

Lutein means: any ingestible form of lutein, as plant extract or oleoresin. It comprises both all-E and Z isomers. Preferably is FloraGLO® Lutein, a Marigold extract containing approx 98% lutein (all-E and Z-isomers) and about 2% of other caotenoids, among them about 0.5-1.5% zeaxanthin. Lutein can be used as suspension in various oils (safflower, corn), or formulated to highly bioavailable beadlets forms, such as the Actilease® FloraGLO® Lutein.

Zeaxanthin means: zeaxanthin and/or its isomers comprising all-E and Z-isomers, as well as SS′, SR (meso-zeaxanthin), and RR′ stereo isomers. Zeaxanthin can be used as suspension in various oils (safflower, corn), or formulated to highly bioavailable beadlets forms, such as Actilease® formulated Optisharp®.

Zeaxanthin means: zeaxanthin and/or commonly used derivatives of zeaxanthin such as meso-zeaxanthin

“Improving/enhancing associative learning” means learning the meaning of signs, symbols, regulations, and how to react to stimuli under trained circumstances, is improved.

“Improving/enhancing learning under stress” means that the subject will experience an improvement in learning new skills or new information when the new task occurs under a stressful condition, such as that experienced as a part of daily life/work stress.

“Improving/enhancing memory under stress” means: the subject's ability to recall information while experiencing a stressful environment or daily life is improved. One example would be recalling answers in an examination situation.

“Improving/enhancing spatial learning or memory” means: the subject's ability to learn the whereabouts of objects in relation to other parts of an environment is enhanced. For example, one might learn one's way around a shopping centre more easily. Another example would be that one would better remember where one parked a car or left one's keys.

“Sole learning-enhancing compounds” means that no other ingredient which is a cognition aid is included in the composition. Examples of ingredients which are specifically excluded include: ginkgo (extracts or individual active ingredients), acetyl-L-carnitine, vitamins C and E, B-Vitamins, citicholine, CoQ10 (CoEnzyme-Q10), ginseng, huperzine A, Omega-3 fatty acids such as DHA/EPA, phosphatidylserine, pine bark extract (Pycnogenol), or Bacopa monieri plant extracts.

“Observing” the enhanced cognitive aspect means either an observer notes the subject has had an improvement, or that the subject him/herself notes that they have had an improvement. These improvements may be quantified using standardized measurements, but they need not be, and can be based on subjective feeling or experience only.

“Treatment” also encompasses co-treatment as well as prevention. “Prevention” is not limited to the complete absence of symptoms in the future, but is intended to include:

lessening of the risk that an individual or a population will exhibit a symptom, lessening the symptoms associated with a particular condition, decreasing the time of onset of a particular condition, lessening the severity of a condition, and decreasing the likelihood that an asymptomatic individual will show a condition in the future.

To achieve these improvements, administration over several days (for example at least six or ten days) is recommended, and administration daily for several weeks is generally preferred.

In preferred embodiments, the lutein or lutein plus zeaxanthin is ingested by a healthy subject. By “healthy”, it is meant that the subject is not suffering from any conditions which impair his/her mental health, i.e. is not suffering from conditions characterized by a deterioration in memory such as dementia, Alzheimer's disease or the like, depression, or other psychotic conditions which affect memory and learning such as schizophrenia.

Thus, another aspect of this invention would be the use of lutein or lutein and zeaxanthin to improve associative memory.

Another aspect of this invention would be the use of lutein or lutein and zeaxanthin to improve memory while the subject is under stress. Subjects with improved memory under stress will observe at least one of the following:
Not forgetting to perform an assigned task at work (when several tasks are pending)
Remembering facts, etc. during public speaking
Better performance under examination conditions or conditions where a speedy recall is required.

Another aspect of this invention would be the use of lutein or lutein and zeaxanthin to improve spatial memory. Subjects with improved spatial memory will observe at least one of the following:
Fewer “misplaced” items such as one's keys, glasses, mobile phone or TV remote control
Ability to easily orient oneself in the surrounding environment such as in a house, while driving or simply remembering where one parked the car in a large parking lot.

Aside from applications for humans, the compositions of this invention have additional uses in the veterinary world. Conditions under which animals would benefit are particularly training procedures and education for specific purposes, eg for hunting dogs, guide dogs, police dogs etc, or animals used in movie industry. Animals which can benefit from enhanced cognitive function include those animals which are subject to stressful conditions. Such conditions occur, for example, after capture or transport or may be due to housing conditions (such as change of domicile or owner), when the animals develop analogous disorders and are distressed or aggressive, or display stereotypic behaviour, or anxiety and obsessive-compulsive behaviour. Animals which are subject to stress would also include those which are racing animals (e.g. dogs, horses, camels), or used in various sports, performing animals (such as circus animals and those appearing on stage, television or in the movies) and horses which perform dressage and other highly disciplined routines.

Preferred “animals” are pets or companion animals and farm animals. Examples of pets are dogs, cats, birds, aquarium fish, guinea pigs, (jack) rabbits, hares and ferrets. Examples of farm animals are aquaculture fish, pigs, horses, ruminants (cattle, sheep and goats) and poultry.

### Nutraceutical Uses/Formulations/Dosages

The term “nutraceutical” as used herein denotes usefulness in both nutritional and pharmaceutical fields of application. Thus, novel nutraceutical compositions can be used as supplements to food/feed and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

Moreover, a multi-vitamin and mineral supplement may be added to nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions according to the present invention may be in any galenic form containing a conventional carrier material that is suitable for administering to the body, especially in any form that is conventional for oral administration, e.g. in solid forms such as (additives/supplements for) food or feed, food or feed premix, fortified food or feed, tablets, pills, granules, dragées, capsules and effervescent formulations, such as powders and tablets, or in liquid forms, such as solutions, emulsions or suspensions as e.g. beverages, pastes and oily suspensions. The pastes may be incorporated in hard- or soft-shell capsules, whereby the capsules feature e.g. a matrix of (fish, swine, poultry, cow) gelatine, plant proteins or ligninsulfonate. Examples for other application forms are those for transdermal, parenteral or injectable administration. The dietary and pharmaceutical compositions may be in the form of controlled (delayed) release formulations.

Examples of food are dairy products including, for example, margarines, spreads, butter, cheese, yoghurts or milk-drinks

Examples of fortified food bread, cereal bars, bakery items, such as cakes and cookies, and potato chips or crisps.

Examples of pet foods are wet canned foods, treats, and pellets.

Beverages encompass non-alcoholic and alcoholic drinks as well as liquid preparations to be added to drinking water and liquid food. Non-alcoholic drinks are e.g. soft drinks, sports drinks, fruit juices, lemonades, teas and milk-based drinks Liquid foods are e.g. soups and dairy products. The nutraceutical composition containing lutein or lutein plus zeaxanthin may be added to a soft drink, an energy bar, or a candy, such that an adult consumes up to 2 mg/kg body weight per day.

If the nutraceutical composition is a pharmaceutical formulation the composition further contains pharmaceutically acceptable excipients, diluents or adjuvants. Standard techniques may be used for their formulation, as e.g. disclosed in Remington's Pharmaceutical Sciences, 20th edition Williams & Wilkins, Pa., USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives.

For animals including humans a suitable daily dosage of lutein or lutein plus zeaxanthin, for the purposes of the present invention, may be within the range from 0.15 mg per kg body weight to about 10 mg per kg body weight per day.

The following non-limiting Examples are presented to better illustrate the invention.

### Example 1

### Effects of Lutein in a Model of Neurotoxicity In Vitro

Glutamate is one of the principal excitatory amino acid in the central nervous system, and as such plays a crucial role in normal physiology. Perturbations in normal regulatory mechanisms of glutamate functions may lead to excessive, neurotoxic activation of glutamate receptors in the brain, which may be detrimental to learning and memory performance in the normal and aging brain. Using a primary culture of neurons, these conditions can be reproduced by glutamate intoxication. Impact on cultured neurons was indicated by morphological changes in the neurofilament cytoskeleton network of the neurons.

Neuroprotective effects of single compounds and combinations of compounds were evaluated by analyzing network density using labelling with an anti-neurofilament antibody (heavy chain 200 kD) specific to mature neuron neurites.

Riluzole (a pharmacological neuroprotective agent) and MK801 were used as inhibitory compounds counteracting neurotoxic effects of glutamate resulting in outgrowth of neurites. Inhibitory MK801 (complete blockage of NMDA-type glutamate receptors) was powerful and led to 100% recovery after intoxication.

Neuroprotective functions of lutein were tested using the schedule shown below:

| | |
|---|---|
| Day 0 | Cell Seeding |
| Day 1 | Medium Addition |
| Day 2 | Medium Change |
| Day 5 | Medium changed and Compounds added |
| Day 7 | Medium changed and Compounds added |
| Day 9 | Medium changed and Compounds added |
| Day 12 | Glutamate intoxication |
| Day 12 + 6 | Analysis |
| hours | |

On day 5, the culture medium was replaced by control culture medium or medium containing lutein (3 concentrations) within physiological range, n=6 per concentration). Half of the medium was changed every 2-3 days. On day 12, cells were intoxicated with 250 μM of glutamate for 6 hours.

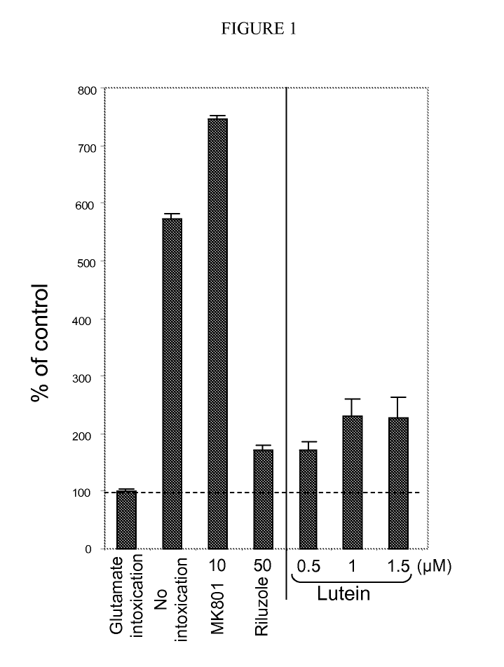

Neurite length of control cultures (glutamate intoxication without any additional compound) was set as 100%. These experiments showed that lutein pre-treatment (all concentrations used, p<0.05 and <0.01) significantly protected neurons from excitotoxic glutamate neuro-toxicity (see FIG. 1)

### Example 2

### Effects of Lutein and/or Zeaxanthin in a New, Totally Automated, Rodent Model of Learning and Memory

The cognitive performance of mice treated with lutein and/or zeaxanthin was compared with that of vehicle-treated, age-matched controls in the IntelliCage®, a system which enables automated monitoring of spontaneous and learning behavior of mice in a homecage-like environment (NewBehavior AG, Zurich, Switzerland, www.newbehavior.com; Galsworthy et al. 2005, Behav Brain Res 157: 211-217; Onishchenko et al. 2007, Toxicol Sci 97: 428-437, Mechan et al., 2009, J. Neurosci. Methods, 180:43-51). Individual mice are recognized by sensors within the IntelliCage corners reading a transponder (reference identification tag) which is implanted into the scruff of the mouse's neck.

Each IntelliCage® is essentially a large cage (37.5×55×20.5 cm), into which is placed a metal frame, comprising four recording (operant) chambers. The recording chambers fit into the corners of the cage, each covering a 15×15×21 cm right-angled triangular area of floor space. In-cage antennae enable automatic monitoring of each individual mouse's corner visits; photo-beams within each corner enable automated recording of individual nosepokes and licks of the water bottle spouts. Four triangular mouse shelters are placed in the center of the cage, above which is situated a food hopper, enabling ad libitum access to food.

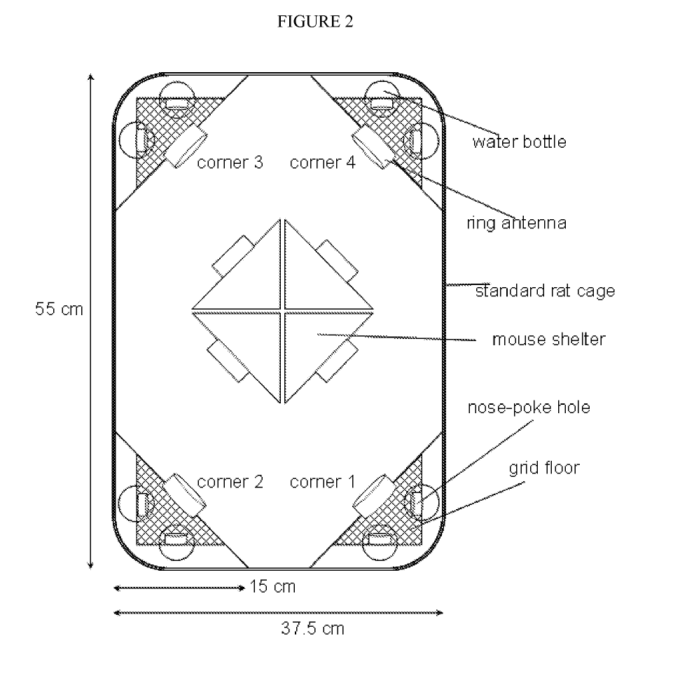

Each recording chamber comprises: (1) a plastic ring (30 mm inner diameter) which serves as an entrance into the chamber and houses the circular antenna which registers corner visits; (2) a grid floor, which the mice sit on once they have entered the chamber; (3) two circular openings (13 mm diameter) which enable access to water bottle spouts; each opening is crossed by photo-beams which register nose-pokes; (4) two motorized doors, which allow (door open) or prohibit (door closed) access to the water bottle spouts; (5) two water bottles; (6) tubing, through which air-puffs can be delivered as aversive stimulation; (7) different colored light diodes, which can be used for conditioning experiments (Mechan et al., 2009 J Neurosci Methods 180:43-51). See FIG. 2.

### Experimental Phase:

The study included two test groups (n=12-14 per group): vehicle (control), Lutein (9 mg/kg BW). (FloraGLO® Lutein, a Marigold extract containing approx 98% lutein (all-E and Z-isomers) and about 2% of other caotenoids, among them about 1-1.5% zeaxanthin.

Lutein was included in the normal animal chow. All mice were administered test substances or placebo in the feed (50 mg/kg feed) 12 days prior to the IntelliCage study and throughout the 4 week study.

During an initial adaptation period (9 days) mice had free access to all corners, water and feed and could freely explore the cage. Subsequently, mice had to learn to apply nose-pokes (nose-poke adaptation module, 2 days); all doors were initially closed (access to water was prohibited) and mice had to perform a nose-poke in order to open a door and to reach a water bottle spout. Data collected comprised several parameters, such as the least-preferred corner of each individual mouse, which was noted for programming the next modules.

After nosepoke adaptation the following tasks were performed:
side discrimination paradigm: a test of associative learning and memory and memory and attention
associative memory paradigm: a test of associative learning and memory with negative reinforcement (stress)
place learning paradigm: a test of spatial learning and memory and orientation

### Side Discrimination Paradigm

This module was designed to test attention and associative memory. One correct corner was assigned to each mouse. In this corner only one side (of two) was assigned as correct, and was indicated to the animals by a green LED. At the correct side animals could make a nosepoke and subsequently drink from the water bottle. During this module the place errors (i.e. percentage of visits to incorrect corners) and side errors (i.e. percentage of nosepokes at the incorrect side of the correct corner) were recorded.

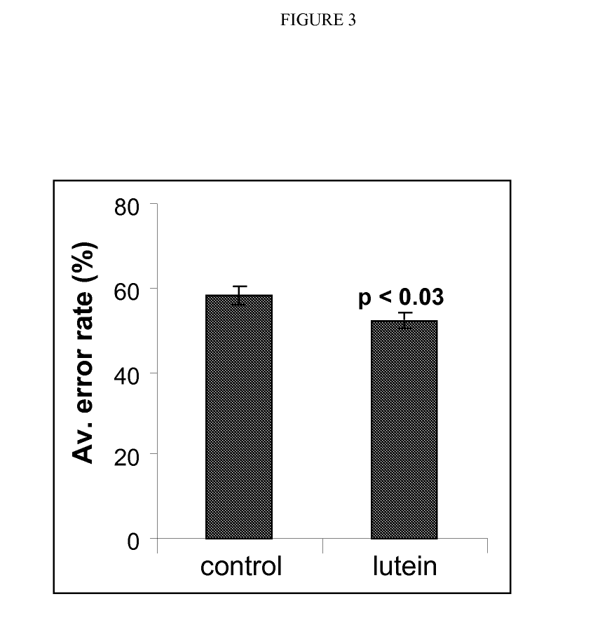

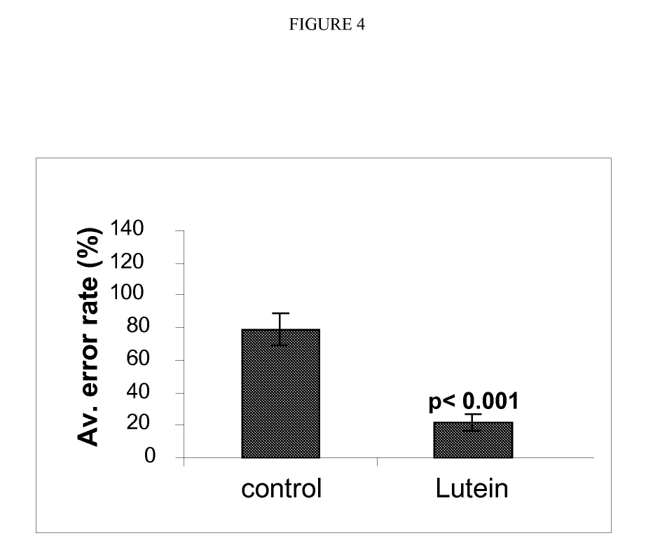

These data indicate improved attention following chronic treatment with lutein since both the place error rate (FIG. 3) and side error rate (FIG. 4) were significantly lower than those of the vehicle-group during the active phase of the test.

In summary, results from the automated IntelliCage® studies show that treatment with lutein results in a significant improvement of learning and memory in mice to associate the presence of a light signal for a short period (2 sec) with the correct corner and the correct side of that corner in order to access to water, when compared to vehicle-treated littermates.

### Associative Memory Paradigm

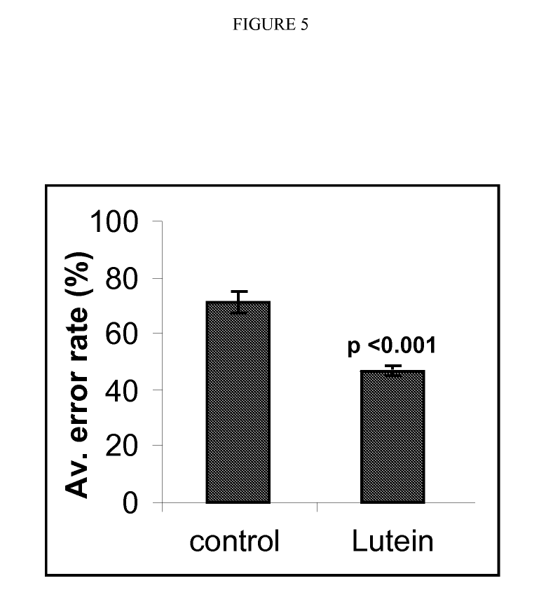

In this module, a correct corner was assigned to each mouse and was indicated by the presentation of a light for 3 s. Thus, a nosepoke in the correct corner opened the doors and mice had access to water within that particular corner, while each visit to any incorrect corner resulted in an air puff punishment (stress). Results are shown in FIG. 5, indicating that mice treated with lutein performed significantly better to learn and memorize the place to receive water (and consequently avoid punishment) in comparison to vehicle-treated littermates.

### Place Learning Paradigm

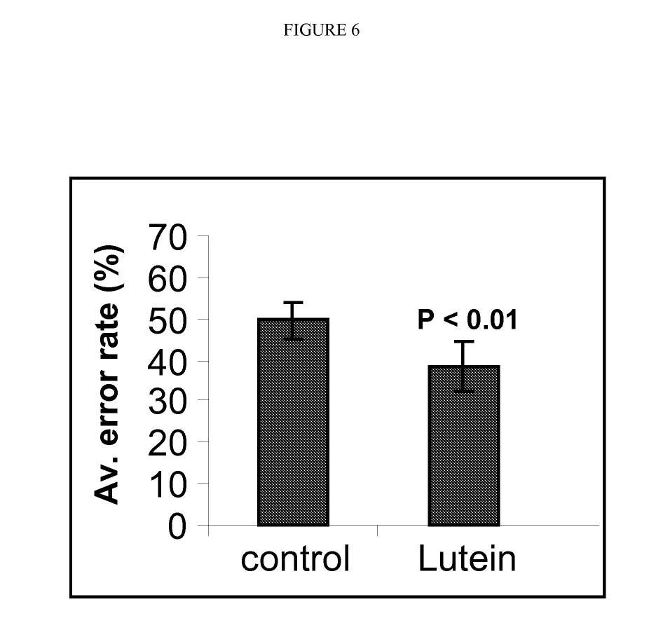

In this learning task mice had to enter the correct corner and perform a nosepoke at either side of the corner in order to get access to water. Corners were assigned according to the animals' visiting behavior during the previous nosepoke adaptation module, the least preferred corner being assigned as the correct corner for each mouse if applicable. No additional signal (such as light) was provided, thus directing the mice to try to learn to memorize the position of the corner to access water based on spatial cues in their surroundings and/or cages. FIG. 6 summarizes the results of this test showing that lutein-treated mice perform significantly better than their placebo-fed littermates.

In summary, results from the automated IntelliCage® studies confirm that treatment with lutein for several weeks results in a significant improvement of various types of learning and memory in mice when compared to vehicle-treated littermates.

In a second set of experiments, varying doses of lutein (8 mg/kg/d, 16 mg/kg/d and 30 mg/kg/d) or a combination of lutein and zeaxanthin (15 and 16 mg/kg/d, respectively) were tested in the above-described learning and memory paradigms. Animals supplemented with the intermediate lutein dose exhibited a significant improvement of performance in the associative memory task compared with the control group, with respect to both place errors and side errors. Moreover, animals fed with lutein and zeaxanthin had a superior performance with respect to side errors.

These experiments were run in animals that were 2-3 months younger than the first set of experiments suggesting that the beneficial lutein effects may vary depending on the age and cognitive paradigm used.

### Example 3

### Preparation of a Soft Gelatine Capsule

A soft gelatine capsule may be prepared comprising the following ingredients:

| Ingredient | Amount per Capsule |
|---|---|
| Lutein (eg FloraGLO ® | 10 mg |
| Lutein 20% Corn Oil) | |
| from DNP AG, | |
| Kaiseraugst, Switzerland | |
| Zeaxanthin (contained in | 0.25 mg |
| FloraGLO ® Lutein 20% | |
| Corn Oil) | |
| Lecithin | 50 mg |
| Corn oil | 50 mg |
| Soybean oil | 189.75 mg |

Two capsules per day for 3 months may be administered to a human adult.

### Example 4

### Preparation of an Instant Flavoured Soft Drink

| Ingredient | Amount [g] |
|---|---|
| FloraGLO ® Lutein 10% CWS/S-TG | 0.480 |
| from DNP AG, Kaiseraugst, Switzerland | (48 mg lutein) |
| Sucrose, fine powder | 763 |
| Ascorbic acid, fine powder | 2.0 |
| Citric acid anhydrous powder | 55.0 |
| Lemon flavour | 8.0 |
| Trisodium citrate anhydrous powder | 6.0 |
| Tricalciumphosphate | 5.0 |
| β-Carotene 1% CWS from DNP AG, | 0.4 |
| Kaiseraugst, Switzerland | |
| Total | 840 |

All ingredients are blended and sieved through a 500 μm sieve. The resulting powder is put in an appropriate container and mixed in a tubular blender for at least 20 minutes. For preparing the drink, 840 g of the obtained mixed powder are mixed with sufficient water to produce one litre of beverage.

The ready-to-drink soft drink contains ca. 12 mg of lutein per serving (250 ml). As a strengthener and for general well-being 1 servings per day (250 ml) may be drunk. Memory under stress is seen to improve.

### Example 5

### Preparation of a Fortified Non-Baked Cereal Bar

| Ingredient | Amount [g] |
|---|---|
| FloraGLO ® Lutein 10% | 0.4 |
| CWS/S-TG from DNP AG, | |
| Kaiseraugst, Switzerland | |
| (containing 40 mg lutein) | |
| OPTISHARP (Zeaxanthin) | 0.16 |
| 5% CWS/S-TG from DNP | |
| AG, Kaiseraugst, Switzerland | |
| (containing 8 mg zeaxanthin) | |
| Salt | 1.5 |
| Glucose syrup | 130.0 |
| Invert sugar syrup | 95.0 |
| Sorbitol Syrup | 35.0 |
| Palm kernel fat | 60.0 |
| Baking fat | 40.0 |
| Lecithin | 1.7 |
| Hardened palm-oil | 2.5 |
| Dried and cut apple | 63.0 |
| Cornflakes | 100.0 |
| Rice crispies | 120.0 |
| Wheat crispies | 90.0 |
| Roasted hazelnut | 40.0 |
| Skimmed milk powder | 45.0 |
| Apple flavour 74863-33 | 2.0 |
| Citric acid | 5.0 |
| Total amount | 795 |

The lutein and zeaxanthin is premixed with skimmed milk powder and placed in a planetary bowl mixer. Cornflakes and rice crispies are added and the total is mixed gently. Then the dried and cut apples are added. In one cooking pot, water and salt are mixed in the amounts given above (solution 1). In a second cooking pot, glucose-, invert sugar- and sorbitol-syrups are mixed in the amounts given above (solution 2). The fat phase constitutes a mixture of baking fat, palm kernel fat, lecithin and emulsifier. Solution 1 is heated to 110° C. Solution 2 is heated to 113° C. and then cooled in a cold water bath. Subsequently, solutions 1 and 2 are combined. The fat phase is melted at 75° C. in a water bath, then added to the combined mixture of solutions 1 and 2. Apple flavour and citric acid are added to the liquid sugar/fat mix. The liquid mass is added to the dry ingredients and mixed well in the planetary bowl mixer. The mass is put on a marble plate and rolled to the desired thickness. The mass is cooled down to room temperature and cut into pieces. The non-baked cereal bar contains ca. 10 mg lutein and 2 mg zeaxanthin per serving (20 g).

### Example 6

### Dry Dog Feed Containing Lutein

A commercial basal diet for dogs (e.g. Mera Dog “Brocken”, MERA-Tiernahrung GmbH, Marienstraβe 80-84, D-47625 Kevelaer-Wetten, Germany) is sprayed with a solution of lutein, lutein, eg. FloraGLO® Lutein 5% Corn Oil together with antioxidants such as vitamin C (e.g. ROVIMIX® C-EC from DSM Nutritional Products Ltd, Kaiseraugst, Switzerland) or its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer approx. 5 mg lutein per kg food. For heavier dogs, the feed mix is prepared accordingly.

### Example 7

### Wet Cat Food Containing Lutein

A commercial basal diet for cats (e.g. Happy Cat “Adult”, Tierfeinnahrung, Siidliche Hauptstraβe 38, D-86517 Wehringen, Germany) is mixed with a solution of lutein, eg. FloraGLO® Lutein 5% Corn Oil together in water, together with antioxidants such as vitamin C (e.g. ROVIMIX® C-EC from DSM Nutritional Products Ltd, Kaiseraugst, Switzerland) or its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to a cat a daily dose of 3 mg lutein per kg food. The food composition is dried to contain dry matter of about 90% by weight.

1. A method of enhancing an aspect of memory in a healthy individual, wherein the aspect of memory is selected from the group consisting of: associative memory, spatial memory and memory under stress comprising:
administering a composition consisting of: a) an effective amount of either lutein or the combination of lutein and zeaxanthin; and b) an appropriate carrier;
and observing the enhanced associative memory, spatial memory or memory under stress.

2. A method of enhancing learning and memory under stress comprising:
administering a composition consisting of: a) a stress related learning and memory enhancing effective amount of either lutein or the combination of lutein and zeaxanthin; and b) an appropriate carrier;
and observing the enhanced learning and memory under stress.

3. A method of enhancing spatial learning and memory comprising:
administering a composition consisting of: a) a spatial related learning and memory enhancing effective amount of either lutein or the combination of lutein and zeaxanthin; and b) an appropriate carrier;
and observing the enhanced spatial learning and memory.

4. The use of lutein or the combination of lutein and zeaxanthin as the sole active compounds to enhance a) associative learning and memory, b) learning and memory under stress or c) spatial learning and memory.

5. The use of lutein or the combination of lutein and zeaxanthin as the sole active compound(s) which are active in respect to enhancing memory, to make a nutraceutical or functional food which enhances a) associative learning and memory, b) learning and memory under stress or and/or c) spatial learning and memory.
